SEQUENCE LISTING (11 sequences)

SEQ ID NO: 1 - DNA, 1329 bp, Arabidopsis thaliana
atggtaactagagaaacgaagttgacgtcagagcgagaagtagagtcgtccatggcgcaagcgagacataatggaggaggtggtggtgagaatcatccgtttacttctttgggaagacaatcctctatctactcattgacccttgacgagttccaacatgctttatgtgagaacggcaagaactttgggtccatgaacatggacgagtttcttgtctctatttggaacgcagaggagaataataacaatcaacaacaagcagcagcagctgcaggttcacattctgttccggctaatcacaatggtttcaacaacaacaataacaatggaggcgagggtggtgttggtgtctttagtggtggttctagaggcaacgaagatgctaacaataagagagggatagcgaacgagtctagtcttcctcgacaaggctctttgacacttccagctccgctttgtaggaagactgttgatgaggtttggtctgagatacatagaggtggtggtagcggtaatggaggagacagcaatggacgtagtagtagtagtaatggacagaacaatgctcagaacggcggtgagactgcggctagacaaccgacttttggagagatgacacttgaggatttcttggtgaaggctggtgtggttagagaacatcccactaatcctaaacctaatccaaacccgaaccaaaaccaaaacccgtctagtgtaatacccgcagctgcacagcaacagctttatggtgtgtttcaaggaaccggtgatccttcattcccgggtcaagctatgggtgtgggtgacccatcaggttatgctaaaaggacaggaggaggagggtatcagcaggcgccaccagttcaggcaggtgtttgctatggaggtggcgttgggtttggagcgggtggacagcaaatgggaatggttggaccgttaagcccggtgtcttcagatggattaggacatggacaagtggataacataggaggtcagtatggagtagatatgggagggctaaggggaaggaaaagagtagtggatggtccagtggagaaagtagtggagagaagacagaggaggatgatcaagaaccgcgagtctgctgctagatctagagcaagaaaacaagcatatacagtggaattggaagctgaacttaaccagttgaaagaagagaatgcgcagctaaaacatgcattggcggagttggagaggaagaggaagcaacagtattttgagagtttgaagtcaagggcacaaccgaaattgccgaaatcgaacgggagattgcggacattgatgaggaacccgagttgtccactctaa

SEQ ID NO: 2 - DNA, 1029 bp, Hordeum vulgare
atggatcggtacgaggtggtgagggacatcgggtcagggaacttcggggtggcgaagctggtgcgggacgtcaggaccagggagcacttcgccgtcaagttcatcgagcgaggccacaagattgatgaacatgttcaaagggagattatgaaccatcggtcactcaagcatccaaatattattcgattcaaggaggtagtgctaactcccacacatttggcaatagttatggaatatgcctctggcggtgagctttttcaaaggatttgcaatgcagggagattcagcgaggatgagggaaggttcttcttccaacaactgatttctggagtgagctattgtcactcgatgcaagtatgtcatagagatttgaaactagagaatactctcttggatggtagtgtcgctcctcgactcaagatttgtgacttcggttactccaagtcttctgtcttgcactctcaaccgaagtcgactgttggcacaccagcatacatcgctccggaggtcctctctagaagagaatatgatggaaaggtcgctgatgtttggtcttgtggagtaacgctctatgtcatgctcgtcggggcatatcctttcgaggaccctgatgagccgaggaacttccgcaaaacgatcgcaaggatactcagcgtacagtactccgttccggactacgttcgagtctcgatggattgcatacatctgctgtcccgcattttcgttggaaatcctcagcagcgaataaccatcccggagatcaagaaccatccatggttcctcaaacgcctgcccgttgagatgacagacgagtaccaaaggggcatgcagttggcagacatgaacacgccgtcgcagagcctggaagaagccatggcgatcatccaggaggcgcagaaaccgggtcataacgccctagggcttgctgggcaggttgcctgcctggggagcatggatctagacgacattgatttcgacgtcgacgacattgacattgagaacagcggggacttcgtgtgcccgttgtga

SEQ ID NO: 3 - DNA, 66 bp, Artificial sequence
  PCR Primer for PKABA1
cgactcgagatgtatccatatgacgtgccggactacgcctccctcatggatcggtacgaggtggtg

SEQ ID NO: 4 - DNA, 30 bp, Artificial sequence
  PCR Primer for PKABA1
cgaactagttcacaacgggcacacgaagtc

SEQ ID NO: 5 - protein, 12 aa, Artificial sequence
  Amino acid sequence for HA
MYPYDVPDYASL

SEQ ID NO: 6 - DNA, 66 bp, Artificial sequence
  PCR Primer for cloning ABI5
cgactcgagatgtatccatatgacgtgccggactacgcctccctcatggtaactagagaaacgaag

SEQ ID NO: 7 - DNA, 28 bp, Artificial sequence
  PCR Primer for cloning ABI5
cgaactagtttagagtggacaactcggg

SEQ ID NO: 8 - DNA, 24 bp, Artificial sequence
  PCR Primer for ABI5
atggtaactagagaaacgaagttg

SEQ ID NO: 9 - DNA, 25 bp, Artificial sequence
  PCR Primer for ABI5
ttagagtggacaactcgggttcctc

SEQ ID NO: 10 - protein, 354 aa, Artificial sequence
  Amino acid sequence of HA-PKABA1
MYPYDVPDYASLMDRYEVVRDIGSGNFGVAKLVRDVRTREHFAVKFIERGHKIDEHVQREIMNHRSLKHPNIIRFKEVVLTPTHLAIVMEYASGGELFQRICNAGRFSEDEGRFFFQQLISGVSYCHSMQVCHRDLKLENTLLDGSVAPRLKICDFGYSKSSVLHSQPKSTVGTPAYIAPEVLSRREYDGKVADVWSCGVTLYVMLVGAYPFEDPDEPRNFRKTIARILSVQYSVPDYVRVSMDCIHLLSRIFVGNPQQRITIPEIKNHPWFLKRLPVEMTDEYQRGMQLADMNTPSQSLEEAMAIIQEAQKPGHNALGLAGQVACLGSMDLDDIDFDVDDIDIENSGDFVCPL

SEQ ID NO: 11 - DNA, 1029 bp, Artificial sequence; identical to SEQ ID NO: 2
  Nucleotide sequence of HA-PKABA1

(19) United States
(12) Patent Application Publication
(10) Pub. No.: US 2010/0083405 A1
(43) Pub. Date: Apr. 1, 2010

(54) Method For Controlling Seed Germination In Non-Dormant Seeds And Use Thereof

(76) Inventors: Luis Lopez-Molina, Geneva (CH); Urszula Piskurewicz, Geneva (CH)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/565,118

(22) Filed: Sep. 23, 2009

Related U.S. Application Data
(60) Provisional application No. 61/100,292, filed on Sep. 26, 2008.

(51) Int. Cl. A01H 5/00; C12N 15/82; C07H 21/04; C12N 15/63; C12N 5/10; A01C 1/00

(52) U.S. Cl. 800/290; 800/278; 800/298; 536/24.1; 435/320.1; 435/419; 47/58.1 SE

(57) ABSTRACT

The present invention is directed to a method of controlling seed germination in non-dormant seeds. The invention is notably directed to a method of delaying germination or temporarily inducing dormancy in non-dormant seeds. The invention further relates to transgenic plants and seeds presenting controlled or delayed germination, notably under environmental favorable conditions.

### CROSS REFERENCE TO RELATED APPLICATION

This application claims the benefit of U.S. Provisional Application Ser. No. 61/100,292, filed Sep. 28, 2008, the disclosure of which is hereby incorporated by reference in its entirety, including all figures, tables and amino acid or nucleic acid sequences.

### FIELD OF THE INVENTION

The present invention is directed to a method of controlling seed germination in non-dormant seeds. The invention is notably directed to a method of delaying germination and/or repressing germination and/or temporarily inducing a desired rate of germination, and/or level of dormancy in non-dormant seeds. The invention further relates to transgenic plants, seedlings and seeds presenting controlled or delayed germination, notably under environmental favorable conditions and related methods of production thereof.

### BACKGROUND OF THE INVENTION

Mature seeds are the endpoint of embryogenesis and seed germination is the developmental process by which a plant abandons its embryonic state to initiate the vegetative phase of its life cycle.

Seeds are adapted to survive for periods of time under adverse conditions until conditions favorable for seedling establishment are encountered. When the grain reaches maximum size, about few weeks after flowering, there follows a net loss of water from the grain and the ripening process begins. Usually, mature seeds present embryos in a quiescent (dormant) and highly resistant desiccated state, reduced metabolic activity, accumulated protective substances to help them to survive under rather severe conditions (e.g. desiccation tolerance), such as food stores that will fuel seed germination (Kroj et al., 2003, Development 130, 6065-6073). In the course of seed maturation, the accumulation of storage products, the suppression of precocious germination, the acquisition of desiccation tolerance, and often the induction of dormancy occur (Bewley and Black, 1994, Seeds. Physiology of Development and Germination. Second Edition. Plenum Press). Seeds become then quiescent at desiccation and can often be stored for a long time.

When seeds are non-dormant, as in this study, imbibition by water is sufficient to trigger germination and start a new lifecycle. Germination commences with the uptake of water by imbibition of the dry seed, followed by embryo expansion and a series of events such as the activation of respiration (Bewley and Black, 1994, above), the repair of macromolecules, reserve mobilization, reinitiation of the cell cycle (Vásquez-Ramos and Sánchez, 2004, Seed Sci Res 13: 113-130), and weakening of covering structures to allow radical protrusion (Groot and Karssen, 1987, Planta, 171:525-531). This usually culminates in rupture of the covering layers and emergence of the radicle, generally considered as the completion of the germination process. At the same time, seeds lose longevity during germination and desiccation tolerance upon radicle protrusion.

In non-endospermic seeds and in Arabidopsis, the mature seed consists of a protective outer layer of dead tissue, the testa (seed coat, diploid maternal tissue), underneath which the endosperm, a single layer of cells, surrounds the embryo (Debeaujon et al., 2000, Plant Physiol. 122, 403-414). Arabidopsis seed germination chronologically involves testa rupture and concomitant endosperm rupture and embryonic axis (i.e. radicle) protrusion (FIG. 1). Rupture events likely involve sugar bond modifying enzymes such as glucanases and mannanases but in Arabidopsis they remain to be identified. Germination is usually defined as visible embryonic axis protrusion out of the testa, i.e. endosperm rupture (Kucera et al., 2005, Seed Science Research 15, 281-307). If conditions are optimal, these steps can be completed 36 h after seed imbibition.

Germination is under tight control by the environment, being affected by light quality, temperature, water potential (i.e. osmotic stress). Environmental factors eventually determine the relative levels of two phytohormones, gibberellins (GA) and abscisic acid (ABA), which exert antagonistic effects on seed germination.

GA and ABA levels tend to be negatively correlated: conditions favorable for seed germination are associated with high GA levels and low ABA levels whereas unfavorable conditions increase ABA levels relative to those of GA. Dry seeds contain endogenous ABA, which plays an essential role during the late stages of seed maturation where it may promote the accumulation of ABI5 mRNA and protein (a basic leucine-zipper transcription factor (TF)), essential to activate the transcription of Late Embryonic and Abundant genes (LEA) and the expression of osmotolerance genes, such as AtEm1 and AtEm6 (Finkelstein and Lynch, 2000, Plant Cell 12, 599-609; Lopez-Molina and Chua, 2000, Plant Cell Physiol. 41, 541-547) which confer osmotolerance and dormancy to the embryo.

When dry seeds lose dormancy, such as after a period of after-ripening, normal germination conditions trigger a decrease in endogenous ABA levels. This leads to a rapid decay in ABI5 mRNA and protein amounts to undetectable levels within 30 hours after imbibition (FIG. 1A). ABA prevents germination and confers osmotolerance by stimulating de novo the accumulation of ABI5. An essential point is that this ABA-dependent seed germination response occurs only within a limited time window of about 48 hours upon imbibition (Lopez-Molina et al., 2002, Plant J., 32, 317-328). However, it must be stressed that ABI5 presence is not sufficient for its activity as shown by experiments using constitutive transgenic ABI5 expression (Lopez-Molina et al., 2001, Proc. Natl. Acad. Sci. USA, 98, 4782-4787).

Under normal conditions (i.e. moisture and light), GA synthesis starts shortly upon seed imbibition, which is essential for the rupture of both testa and endosperm (Lee et al., 2002, Genes Dev. 16, 646-658). In contrast, ABA levels, high in mature seeds, drop rapidly upon imbibition and the role of ABA becomes facultative: after imbibition, a sudden osmotic stress or direct application of ABA (which signals osmotic stresses), efficiently prevents endosperm rupture while delaying that of testa and confers osmotolerance to the arrested embryo.

In non-dormant seeds, moisture is sufficient to trigger germination. However, the environmental conditions encountered by the seed will determine the endogenous levels of GA and ABA, which in turn will define the pace of germination. The possible outcomes for the seed take place between two extreme states: germination is prevented (low GA, high ABA) or unhampered (high GA, low ABA). Prevalent views of how GA and ABA exert their influence to control seed germination emphasize the role of germination repressors.

From an economic point of view, the quality of dry seeds is important in agriculture, since seeds are often the starting material for crop production and crucial for achieving a good harvest.

Normally, grains show some degree of dormancy when harvested and require a period of so-called ‘after-ripening’ before dormancy is broken and germination commences under favorable conditions. However, premature grain germination may occur, whilst still in the ear, when excessive rainfalls occur during growing or harvesting season. Pre-harvest sprouting in mature crops causes a reduction in the quality of the crop in grading and in functional properties. Downgrading of grain quality includes decreasing nutritional properties, severely limiting end-use applications (improper to dough making) and results in lower market prices, causing economic and marketing problems for the grain trade and substantial financial losses to farmers and food processors. Premature germination can also occur during storage which has dramatic economic and human consequences. Methods for sorting pre-harvest-sprouted grain from sound grain have been developed but they are costly and time consuming and do not avoid the loss of part of the harvest product.

Further, the control of seed germination is also an important factor for obtaining quick and uniform germination directly after sowing or planting. Usually, seeds are subjected to “priming”, a process that allows the seeds to absorb enough water to enable their pre-germinative metabolic processes to begin and then arrests them at that stage. The priming process of seeds has the disadvantage that the amount of water absorbed must be carefully controlled as too much would simply allow the seed to germinate and too little would result in the seed ageing. Once the correct amount of water has been absorbed it is then necessary to hold the seed at that water content for a period, typically one to two weeks, before drying it back to the original water content for storage. However, this process is delicate and costly.

Therefore, the development of a method to control seed germination and notably to prevent premature germination in order to preserve seed quality, even in the case of environmental conditions favorable to germination during growing or harvesting season, would be highly desirable.

### SUMMARY OF THE INVENTION

The present invention is directed to a method of controlling seed germination in non-dormant seeds. The invention is notably directed to a method of delaying germination or temporarily inducing a desired rate of germination, and/or level of dormancy in non-dormant seeds. The invention further relates to transgenic plants, seedlings and seeds presenting controlled or delayed germination, notably under environmental favorable conditions and related methods of production thereof.

A first aspect of the invention provides a method of producing a transgenic plant.

A second aspect of the invention provides a method of producing a transgenic seed with delayed germination, as compared to wild-type seed.

A third aspect of the invention provides a method of controlling seed germination.

A fourth aspect of the invention provides a method for expressing nucleotide sequences in a plant.

A fifth aspect of the invention provides a transgenic plant or a transgenic seed, the progeny and propagating material thereof according to the invention.

A sixth aspect of the invention provides an expression cassette, a recombinant vector thereof according to the invention.

A seventh aspect of the invention provides a use of a polynucleotide, an expression cassette or a recombinant vector according to the invention for the production of a seed or a plant.

An eighth aspect of the invention provides a method of producing a crop according to the invention.

A ninth aspect of the invention provides a kit comprising the expression cassette according to the invention and at least one reagent for introducing the expression cassette into a plant cell.

### DESCRIPTION OF THE FIGURES

FIGS. 1A-1C show the early developmental steps upon seed imbibitions. WT Arabidopsis seeds at different times (FIG. 1A) upon imbibition under normal conditions. Testa (FIG. 1B) and endosperm rupture (FIG. 1C) events are indicated by arrows.

FIGS. 2A-2C show the stimulation of ABI5 expression under low GA conditions in Arabidopsis seeds. FIG. 2A: Northern blot analysis of a time course of ABI5 mRNA levels upon WT dry seed (DS) imbibition in absence (Normal) or presence of an inhibitor of GA synthesis (PAC) or ABA as described in Example 1. Hybridization signals can be directly compared between different conditions. Germination percentage (% G) at each time point is indicated. FIG. 2B: Western blot analysis of ABI5 protein levels from the material in A. Signals can be directly compared. FIG. 2C: ABI5 protein levels 48 h after imbibition in gal-3 seeds under normal conditions (gal-3) or with added GA (gal-3+GA) and in PAC-treated WT seeds (PAC) as described in Example 1.

FIGS. 3A-3B show Arabidopsis seed germination repression by stimulation of ABI5 activity for plant material harvested 48 h after seed imbibition under the low GA conditions. FIG. 3A: WT/35S::HA-ABI5 under normal conditions or treated with low PAC concentrations (0.125 μM) that do not prevent WT seed germination, at 72 h (1) and 96 h (2) after seed imbibition. Percentage of germination on low PAC (% G) at 96 are indicated. (a): WT; (b): WT/35S::HA-ABI5. FIG. 3B: Western blot analysis from protein extracts isolated from WT/35S::HA-ABI5 seeds measuring HA-ABI5 protein mobility as described in Example 1.

FIGS. 4A-4D show the effect of co-expression of ABI5 and PKABA1 on Arabidopsis seed germination and ABI5 phosphorylation. FIG. 4A: Schematic map of an estrogen receptor-based transactivator XVE to highly induce HA-PKABA1 (SEQ ID NO:10) expression in transgenic plants. Only regions to be integrated into the plant genome are shown. The vector is a XVE vector (as described in Zuo et al., 2000, above) containing an amplified HA-PKABA1 DNA into the Xho1 and Spe1 restriction sites (not to scale). PG10-90, is a synthetic promoter (Ishige et al., 1999, Plant J., 18, 443-448) controlling XVE; XVE, DNA sequences encoding a chimeric transcription factor containing the DNA-binding domain of LexA (residues 1-87), the transcription activation domain of VP16 (residues 403-479) and the regulatory region of the human estrogen receptor (residues 282-595); TE9, rbcS E9 poly(A) addition sequence; Pnos, nopaline synthase promoter; HPT, hygromycin phosphotransferase II coding sequence; Tnos, nopaline synthase poly(A) addition sequence; OLexA, eight copies of the LexA operator sequence; −46, the −46 35S minimal promoter; MCS, multiple cloning sites for target genes; T3A, rbcsS3A poly(A) addition sequence. Arrows indicate the direction of transcription. FIG. 4B: Western blot analysis of HA-ABI5 and HA-PKABA1 protein levels in WT and WT/35S::HA-ABI5 plants transformed with the ind::HA-PKABA1 DNA construct, under normal conditions in presence (+) and in absence of the inducer (−), as described in Example 2, on protein extracts isolated from plant material 48 h after seed imbibitions. FIG. 4C: Western blot analysis of mobility differences of HA-ABI5 protein isolated from WT/35S::HA-ABI5 lines transformed with a ind::HA-PKABA1 DNA construct, under the indicated germination conditions. FIG. 4D: Plant material used in A at 120 hours after seed imbibitions in normal and excess GA (+) conditions.

FIG. 5 shows the reversibility of the Arabidopsis seed germination blockage after the removal of the inducer on WT plants constitutively expressing HA-ABI5 protein (WT/35S::HA-ABI5) transformed with the ind::HA-PKABA1 DNA construct as described in Example 2.

FIG. 6 shows the expression levels of AtEm6 gene in Arabidopsis by Northern blot analysis of ABI5 mRNA levels in the presence or absence of HA-PKABA1 from plants constitutively expressing HA-ABI5 protein (WT/35S::HA-ABI5) as described in Example 2.

FIG. 7 shows a model for inhibiting seed germination in plants associated with the expression and phosphorylation of ABI5. Plants expressing phosphorylated ABI5 inhibit seed germination by mimicking the response of both GA- and ABA phytohormones under unfavorable conditions for seed germination.

FIGS. 8A-8E show some sequences described in the application and detailed in Table 1. FIG. 8A: SEQ ID NO: 1; FIG. 8B: SEQ ID NO: 2; FIG. 8C: SEQ ID NO: 3, wherein the bold sequence encodes for the His tag. Upstream is the Xho1 restriction site; downstream is the PKABA1 sequence; FIG. 8D: SEQ ID NO:10, wherein the bold amino acids represent the his tag fused to PKABA1 sequence (ORF); FIG. 8E: SEQ ID NO:11.

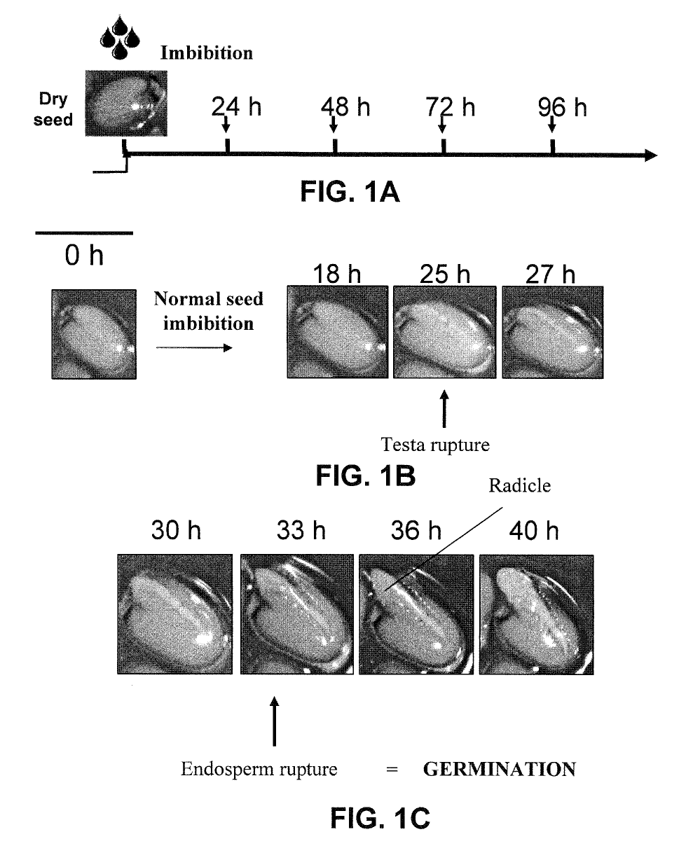

### DETAILED DESCRIPTION OF THE INVENTION

The term “seed, seedling or plant” of the present disclosure is one of which is sensitive to abscisic acid. The term includes all stages in the life of a plant and includes somatic embryos and primed seeds.

The term “crop” comprises crop and other edible plants such as rice, wheat, barley, rye, corn, soybean, and sorghum.

The term “osmotolerance” comprises the tolerance to water (osmotic) stress which is characterized by the measure of a plant's capability to withstand drought or to thrive in large amounts of slats in its water supply.

The term “germination” comprises the developmental process by which a plant abandons its embryonic state to initiate the vegetative phase of its life cycle. Germination is characterized by embryo expansion due to water uptake after imbibitions followed and a series of events such as activation of respiration, repair of macromolecules, reserve mobilization, reinitiation of the cell cycle and weakening of covering structures to allow radicle protrusion. Germination usually culminates in rupture of the covering layers and emergence of the radicle, generally considered as the completion of the germination process. Germination process can be followed for example by the emergence of the radicle tip outside of the testa (outer seed coat).

The term “dormancy” comprises a state characterized by a temporary failure or block of a viable seed to complete germination under physical conditions that normally favor.

The term “delayed germination” comprises a state a viable non-dormant seed characterized by the occurrence of its germination after a longer exposure to physical conditions that normally favor germination in wild-type seeds. Delayed germination includes a state “dormancy-like” state characterized by a temporary failure or block of a viable of complete germination.

The term “non-dormant” characterize a state for a seed characterized by its capacity to germinate over the wide range of normal physical environmental factors possible for the genotype (such as water, oxygen, appropriate temperature, light and/or nitrate etc.).

The term “osmotic stress” comprises the significant changes in water potentials in the environment which can impose osmotic stress to plants, which disrupts its normal cellular activities. Under natural conditions, high salinity and drought are the major causes of osmotic stress to plants.

The term “SnRK2-type kinase” (Sucrose non-fermenting 1-related protein kinase 2 or SnRK2) comprises the SnRK2 kinases and analogs thereof which represents a plant-specific Ser/Thr protein kinase family, a subfamily of sucrose non-fermenting-1 related kinases (SnRKs). The SnRK2 subfamily includes PKABA1 from wheat (Gomez-Cadenas et al. 1999, Proc. Natl. Acad. Sci. USA, 96, 1767-1772; Kobayashi et al., 2004, Plant Cell, 16:1163-1177). The term SnRK2-type kinases includes SnRK2 kinases such as described in Kobayashi et al., 2004 (e.g. SnRK2.2 kinase (AT3G50500) and SnRK2.3 (AT5G66880) kinase).

The term “priming” comprises the treating of plant seeds that enables them to undergo faster and more uniform germination on sowing or planting, with the option of simultaneously treating them with fungicide or other preservatives providing protection during processing or after sowing and allowing their prolonged storage, e.g. in packets displayed at point of sale.

The term “after-ripening” comprises a method used to release dormancy and to promote germination which comprises a period of usually several months of dry storage at room temperature of freshly harvested, mature seeds.

The term “promoter” refers to promoters which promote expression of a DNA molecule and includes constitutive, developmentally regulated promoters, inducible promoters and tissue specific promoters.

The term “constitutive promoter” refers to a promoter which allows for continual transcription of its associated gene. Typically, a constitutive promoter for ABI5 according to the invention is a 35S promoter.

The term “inducible promoter” refers to a promoter which is responsive to an externally administered inducer comprising a chemical or any other stimuli such as an environmental stimulus. In the absence of inducer, the promoter of the second DNA molecule (under iib) is not substantially active: it is either not expressed at all or is expressed at levels which are insufficient to cause significant SnRK2-type kinase expression to block germination. Examples of environmental conditions that may effect transcription by inducible promoters include high salt conditions, wetness conditions, elevated temperature, or application of chemicals/hormones. Exemplary inducible promoters to be used in the context of the invention include estradiol inducible promoters such as XVE promoter as described in Zuo et al., 2000, Plant J., 24, 265-273, stress inducible promoters such as that of RD29a (Party et al., 1994, Plant Cell., 6(11):1567-82) or KIN2 (Kurkela and Borg-Franck, 1992, Plant Mol. Biol., 19(4):689-92) and wetness inducible promoters such as those regulating the ABI4 and RGL2 genes, e.g. CYP707A2 et CYP707A4 gene (Kushiro et al., 2004, Embo J. 23 (7)).

The term “wild-type” refers to a non-transformed plant or seed of the same genus and species.

The term “analog” includes a polypeptide substantially homologous to, but which has an amino acid sequence different from that of native sequence because of one or more deletions, insertions or substitutions. Substantially homologous means an analog amino acid sequence that is at least 60%, at least 65%, at least 70%, at least 80%, at least 85%, at least 90%, at least 95%, at least 96%, at least 97%, at least 98% or at least 99% identical to the native amino acid sequences, as disclosed above. The percent identity of two amino acid or two nucleic acid sequences can be determined by visual inspection and/or mathematical calculation, or more easily by comparing sequence information using a computer program such as Clustal package version 1.83. Examples of variants of variants may comprise a sequence having at least one conservatively substituted amino acid, meaning that a given amino acid residue is replaced by a residue having similar physiochemical characteristics.

### Methods According to the Invention

According to one embodiment, the invention provides a method of producing a transgenic plant, comprising the steps of:

(a) Transforming a plant cell with: (i) a first expression cassette comprising (ia) a promoter operably linked to (ib) a nucleotide sequence encoding for absisic acid insensitive 5 (ABI5); and
(ii) a second expression cassette comprising (iia) an inducible promoter operably linked to (iib) a nucleotide sequence encoding for a Sucrose non-fermenting 1-related protein kinase 2-type (SnRK2-type) kinase;
(b) Regenerating from said transformed plant cell a genetically transformed plant.

According to another embodiment, the invention provides a method of producing a transgenic seed with delayed germination, as compared to wild-type seed comprising the steps of:

(a) Transforming a plant cell with: (i) a first expression cassette comprising (ia) a promoter operably linked to (ib) a nucleotide sequence encoding for ABI5; and a (ii) second expression cassette comprising (iia) an inducible promoter operably linked to (iib) a nucleotide sequence encoding for a SnRK2-type kinase;
(b) Regenerating from said transformed plant cell a genetically transformed plant;
(c) Collecting a population of transgenic seeds from said transgenic plant.

According to another embodiment, the invention provides a method of controlling seed germination, comprising:

(a) providing a seed having a genome comprising (i) a stably integrated expression cassette wherein said expression cassette comprises (ia) a nucleotide sequence encoding for a promoter which is operably linked to (ib) a nucleotide sequence encoding for ABI5 and (ii) a stably integrated expression cassette wherein said expression cassette comprises (iia) a nucleotide sequence of an inducible promoter operably linked to (iib) a nucleotide sequence encoding for a SnRK2-type kinase;
(b) inducing the inducible promoter to block seed germination.

According to another embodiment, the invention provides a method for expressing nucleotide sequences in a plant, the method comprising:

(a) operably linking a first nucleotide sequence to a plant promoter to produce an expression cassette, wherein the nucleotide encodes for ABI5;
(b) operably linking a second nucleotide sequence to an inducible plant promoter to produce an inducible expression cassette wherein the nucleotide encodes for a SnRK2-type kinase; and
(c) generating a transgenic plant comprising the expression cassettes, whereby the first nucleotide sequence is constitutively expressed in the plant and the second nucleotide sequence is expressed upon action of an inducer.

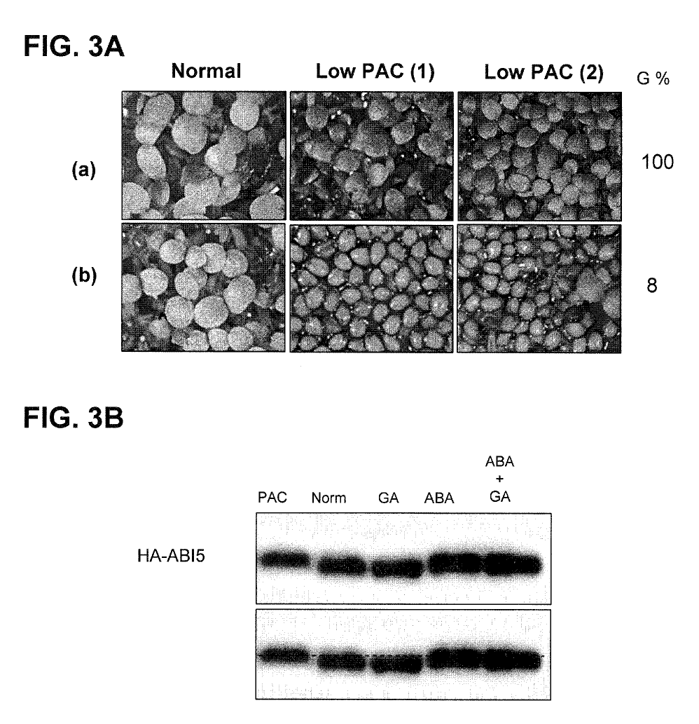

According to a further embodiment, the invention provides a method according to the invention wherein the production of phosphorylated ABI5 in a plant cell is increased as compared to wild-type plant cell through the constitutive increased expression of ABI5 and the induced production of a SnRK2-type kinase as compared to wild-type plant cell by an inducteur. Typically, the production of ABI5 is increased by at least a factor of two and the phosphorylation state of ABI5 is modified, resulting in a mobility shift in SDS-PAGE gels as shown in FIG. 3B.

According to another further embodiment, the invention provides a method according to the invention wherein the promoter under (ia) is a constitutive promoter.

According to another further embodiment, the invention provides a method according to the invention wherein the promoter under (ia) is a constitutive promoter which provides seed preferred expression.

According to another further embodiment, the invention provides a method according to the invention wherein the constitutive promoter under (ia) is the 35S promoter.

According to another further embodiment, the invention provides a method according to the invention wherein the promoter under (iia) is an inducible promoter which provides seed preferred expression.

According to another further embodiment, the invention provides a method according to the invention wherein the inducible promoter under (iia) is an estradiol inducible promoter.

According to another further embodiment, the invention provides a method according to the invention wherein the inducible promoter under (iia) is the estradiol inducible promoter XVE.

According to another further embodiment, the invention provides a method according to the invention wherein the inducible promoter under (iia) is selected from a stress inducible promoter and a wetness inducible promoter.

According to another further embodiment, the invention provides a method according to the invention wherein the nucleotide sequence encoding for ABI5 under (ib) comprises a nucleic acid sequence of SEQ ID NO: 1 (AN AT2G36270).

According to another further embodiment, the invention provides a method according to the invention wherein nucleotide sequence under (iib) encodes for PKABA1 kinase and comprises a nucleic acid sequence of SEQ ID 2: (AN AB058923).

According to another further embodiment, the invention provides a method according to the invention wherein the said plant has delayed germination as compared to wild-type plant.

According to another further embodiment, the invention provides a method according to the invention wherein the plant cell is a plant ovule.

According to another further embodiment, the invention provides a method of producing a transgenic seed according to the invention wherein the method further comprises the steps (d) of screening said population of transgenic seeds for delayed germination as compared to control wild-type seeds and (e) selecting from said population one or more transgenic seeds with delayed germination.

### Seeds, Seedlings or Plants According to the Invention, Progeny and Uses Thereof

According to another embodiment, the invention provides a transgenic plant and the progeny thereof, which comprises (i) a stably integrated expression cassette wherein said expression cassette comprises (ia) a nucleotide sequence encoding for a promoter which is operably linked to (ib) a nucleotide sequence encoding for ABI5 and (ii) a stably integrated expression cassette wherein said expression cassette comprises (iia) a nucleotide sequence of an inducible promoter operably linked to (iib) a nucleotide sequence encoding for a SnRK2-type kinase.

According to another embodiment, the invention provides a transgenic seed comprising a DNA construct capable of (i) constitutively expressing functional ABI5 and of (ii) expressing for a SnRK2-type kinase under induction, at least during the period of seed maturation.

According to a further embodiment, the invention provides a transgenic seed comprising a DNA construct capable of (i) constitutively expressing functional ABI5 and of (ii) expressing for a SnRK2-type kinase under induction, at least during the period of seed maturation, wherein the DNA construct under (i) is a pBA002 vector.

According to a further embodiment, the invention provides a transgenic seed comprising a DNA construct capable of (i) constitutively expressing functional ABI5 and of (ii) expressing for a SnRK2-type kinase under induction, at least during the period of seed maturation, wherein the DNA construct under (ii) is a pER8 vector.

According to another embodiment, the invention provides a transgenic plant or seed obtainable by a method according to the invention.

According to another embodiment, the invention provides an expression cassette comprising a promoter operably linked to (a) a DNA sequence encoding a DNA-binding domain of LexA (residues 1-87); (b) a DNA sequence encoding a transcription activation domain of VP16 (residues 403-479); (c) a DNA sequence encoding a regulatory region of an estrogen receptor (e.g. residues 282-595 form human estrogen receptor) and (d) an amplified PKABA1 DNA into the Xho1 and Spe1 restriction sites, wherein the PKABA1 DNA sequence comprises a silent selectable marker.

According to a further embodiment, the invention provides an expression cassette according to the invention, wherein the amplified PKABA1 DNA is HA-PKABA1 DNA of SEQ ID NO:11.

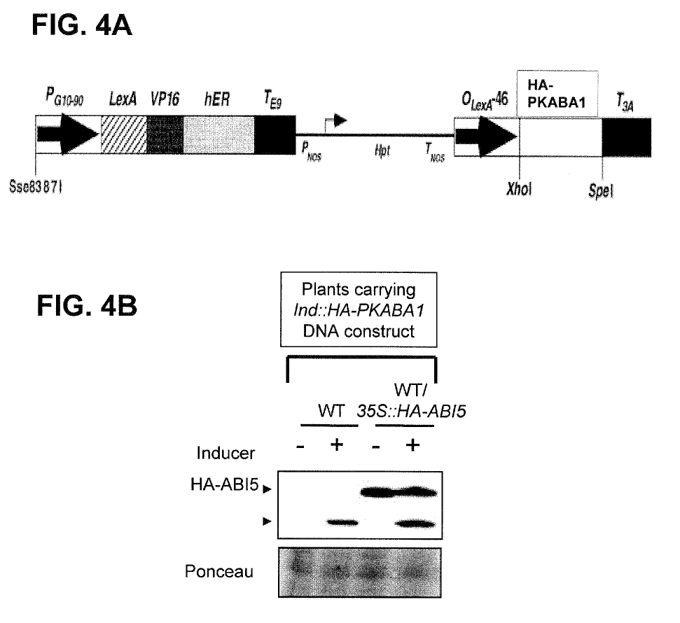

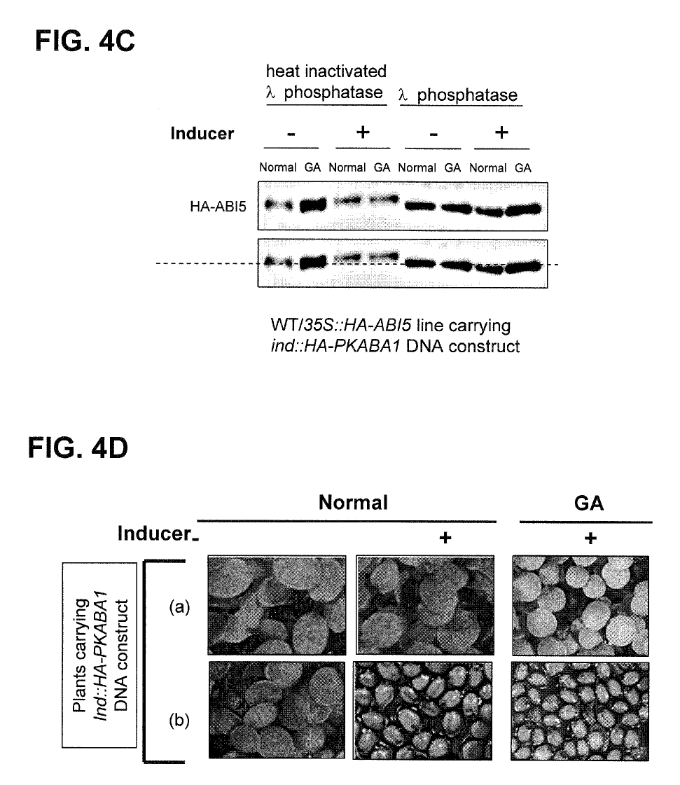

According to a further embodiment, the invention provides an expression cassette according to the invention, wherein the expression cassette is according to FIG. 4A.

According to another embodiment, the invention provides a recombinant vector comprising an expression cassette according to the invention.

A transgenic plant cell comprising an expression cassette according to the invention.

According to another embodiment, the invention provides a transgenic plant comprising a transgenic plant cell according to the invention.

According to another embodiment, the invention provides a progeny or a seed from a plant according to the invention.

According to another embodiment, the invention provides a transgenic seed obtainable by a method according to the invention.

According to another embodiment, the invention provides a propagating plant material derived from a plant according to the invention.

According to another embodiment, the invention provides a use of a recombinant vector according to the invention for the production of a seed or a plant.

According to another embodiment, the invention provides a method of producing a crop, said method comprising the steps of: (a) planting the transgenic plant or a seed according the invention; and (b) harvesting a resulting crop.

According to a further embodiment, the invention provides a method of producing a crop wherein the method further comprises a step (a′) between steps (a) and (b), wherein step (a′) comprises providing an inducteur to the transgenic plant or a seed.

According to a further embodiment, the invention provides a method of producing a crop wherein the provision of an inducteur under further step (a′) is performed by the application to the plant by spraying or by watering the inducteur, optionally in combination with other plant additives such as fertilizers, insecticides, a pesticides, nutrients etc.

According to a further embodiment, the invention provides a crop produced by a method according to the invention.

According to a further embodiment, the invention provides an agricultural product produced by a transgenic plant according or by a transgenic seed according to the invention.

According to another embodiment, the invention provides a kit comprising the expression cassette according to the invention and at least one reagent for introducing the expression cassette into a plant cell.

According to a further embodiment, the invention provides a kit according to the invention, further comprising an expression cassette for overexpressing ABI5. Typically, the expression cassette for overexpressing ABI5 is an expression cassette such as described in Lopez-Molina et al., 2001, above.

According to another further embodiment, the invention provides a method, a transgenic plant, a seedling and a seed wherein the promoter is a seed specific promoter.

According to a further embodiment, the invention includes transgenic plants and plant parts, such as for example, seeds, fruits, leaves, and flowers, comprising the transgenic plant cells. Additionally, the present invention includes agricultural products produced from the transgenic plant cells, plant parts, or plants disclosed herein.

According to a further embodiment, the promoters confer expression of the polynucleotide preferentially in the embryo, endosperm or developing seed of a cereal plant relative to at least one other tissue or organ of said plant.

The objects of the invention are particularly useful in crop plant cells, such as rice, wheat, barley, rye, corn, oats, potato, sweet potato, turnip, squash, pumpkin, zucchini, melon, soybean, and sorghum.

Table 1 below presents the Sequence identity numbers and associated molecules:

TABLE 1

| SEQ ID NO: | Molecule |
|---|---|
| 1 | Nucleotide sequence encoding for ABI5 |
| 2 | Nucleotide sequence encoding for PKABA1 |
| 3 | PCR Primer for PKABA1 |
| 4 | PCR Primer for PKABA1 |
| 5 | Amino acid sequence for HA |
| 6 | PCR Primer for cloning ABI5 |
| 7 | PCR Primer for cloning ABI5 |
| 8 | PCR Primer for ABI5 |
| 9 | PCR Primer for ABI5 |
| 10 | Amino acid sequence of HA-PKABA1 |
| 11 | Nucleotide sequence of HA-PKABA1 |

References cited herein are hereby incorporated by reference in their entirety. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims. Examples illustrating the invention will be described hereinafter in a more detailed manner and by reference to the embodiments represented in the Figures.

### EXAMPLES

The following abbreviations refer respectively to the definitions below:

μM (micromolar), vol. (volume), wt (weight), ABA (abscisic acid), ABI5 (abscisic acid insensitive 5), DS (dry seeds), ER (Endosperm rupture), GA (gibberellins), HA (hemagglutinin), LEA (Late Embryonic and Abundant genes), PAC (paclobutrazol), rRNA (ribosomal RNA), SnRK2 (Sucrose non-fermenting 1-related protein kinase 2), TR (testa rupture), WT (wild type).

### General Procedures & Conditions

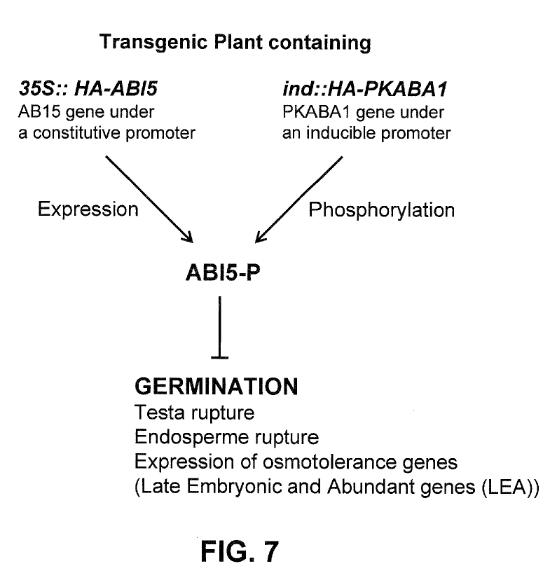

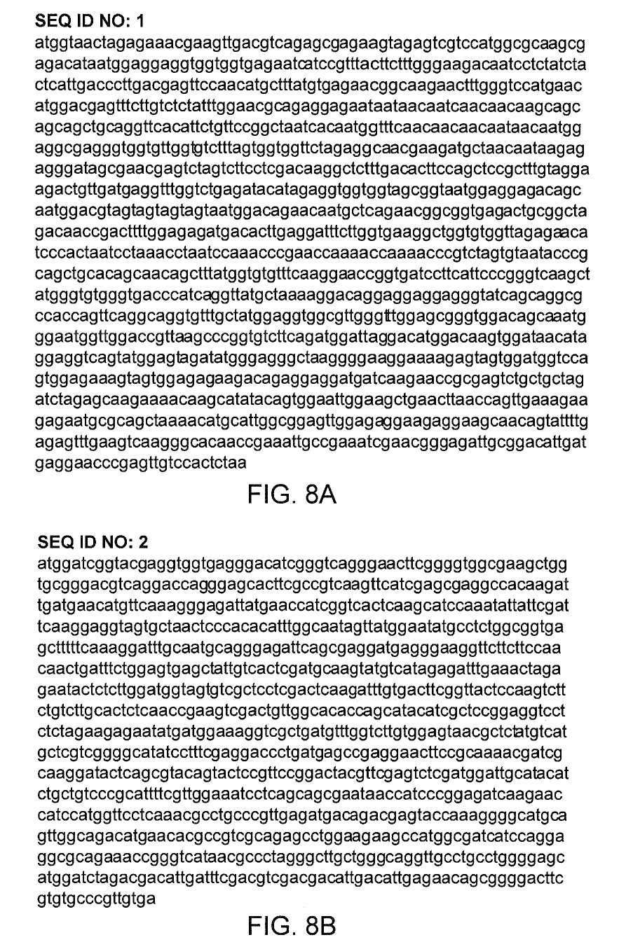

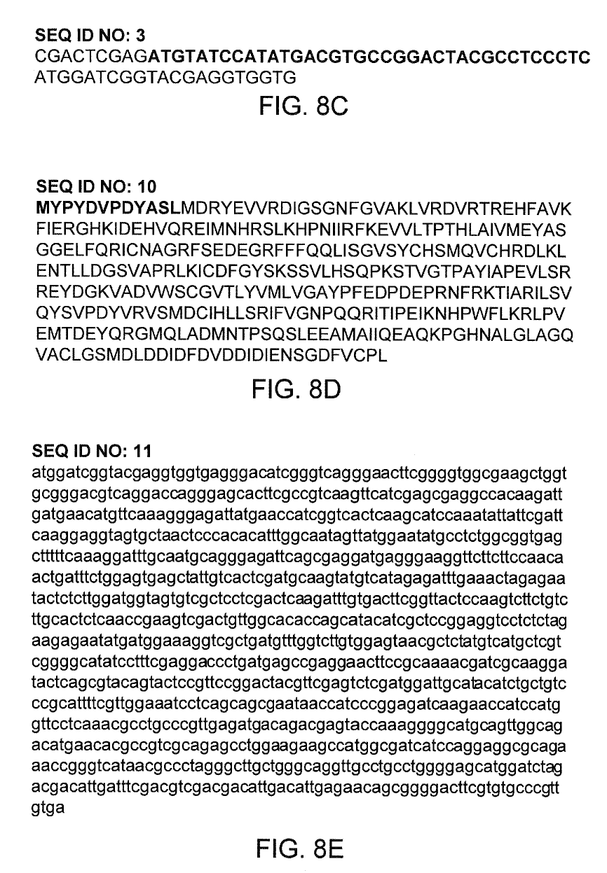

In a particular aspect, the present invention consists of producing a transgenic plant co-expressing ABI5 and a SnRK2-type kinase for controlling and/or inhibiting seed germination in plants as described on FIG. 7. The method according to the invention comprises the expression of ABI5 gene under a constitutive promoter and the expression of a SnRK2-type kinase under an inducible promoter in a plant which will phosphorylate overexpressed ABI5, upon action of the inducer.

Resulting transgenic plants according to the invention express phosphorylated ABI5. The resulting expression of phosphorylated ABI5 (repressing factor) in plants leads to a rapid germination arrest as assessed by the lack of testa and endosperm ruptures and expression of Late Embryonic and Abundant genes (LEA).

### Statistics

Average values were obtained from a minimum of three independent seed batches. Within a seed batch, measurements were at least performed twice giving consistent results. We used the Student's t test (two-tailed assuming unequal variance) to compare average mean values in order to determine if their difference was statistically significant (t<0.05).

### Plant Material

Wild type (WT) Arabidopsis (Columbia ecotype) was used. The transgenic Arabidopsis line (Ler ecotype) constitutively overexpressing and accumulating ABI5 protein fused to hemagglutinin (HA) peptide tag (referred to as WT/35S::HA-ABI5 plants) was generated as described in Lopez-Molina et al., 2001, above. Transgenic Arabidopsis lines were generated using the Agrobacterium tumefaciens vacuum-infiltration method (Bechtold and Pelletier, 1998, Methods Mol. Biol. 82, 259-266). Seeds (T1) from infiltrated plants were plated in selection medium as described (Zuo et al., 2000, above; Lopez-Molina et al., 2001, above). Non-dormant seeds are used in absence of seed stratification procedure. The infiltration in the plant ovule is performed following the standard “Floral Dip” (Arabidopsis protocol Edited by J M Martinez-Zapater and Julio Salina. Humana Press, “In Planta Agrobacterium-Mediated Transformation of Adult Arabidopsis thaliana Plants by Vacuum infiltration”, Page 259).

### Plasmid Constructs and Plant Transformation

DNA manipulations were performed according to standard methods (Sambrook et al., 1989, Molecular cloning: a Laboratory Manual, Cold Spring Harbor, N.Y.: Cold Spring Harbor Laboratory Press). The following primers were used for cloning ABI5:

(SEQ ID NO: 6)

5′CGACTCGAGATGTATCCATATGACGTGCCGGACTACGCCTCCCTCATG
GTAACTAGAGAAACGAAG
and

(SEQ ID NO: 7)

5′CGAACTAGTTTAGAGTGGACAACTCGGG.

### Medium Conditions

When seeds are sown under “normal conditions” it refers to conditions where seeds are imbibed in a standard germination medium and provided with light (germination assay condition below). “ABA conditions” and “low GA conditions” are respectively conditions where ABA (e.g. 5 μM) and paclobutrazol (e.g. 5 μM, PAC), an inhibitor of GA synthesis, are added to the medium.

### Germination Assays

All seed batches compared in this study were harvested on the same day from plants grown side by side (i.e. identical environmental conditions). Dry siliques were obtained about 8 weeks after planting and left for a further 4 weeks at room temperature prior to seed harvesting. Seeds were then permanently stored at 4° C. Seeds obtained in this manner lacked dormancy. A minimum of three independently grown seed batches were used for measuring percent TR and ER. For TR and ER assays, seeds were surface sterilized as described in Lopez-Molina and Chua, 2000, above, and sown in plates with MS medium containing 0.8% (wt/vol.) Bacto-Agar (Applichem). Plates were incubated in a climate-controlled room (20-25° C., 16 h light/day, light intensity 80 ME/m²/s, humidity 70%). Between 100 and 300 seeds were examined with a Stemi 2000 (Zeiss) stereomicroscope and photographed with a high-resolution digital camera (Canon Power G6, 7.1 Megapixels) at different times of seed imbibition. Photographs were enlarged electronically for measurement of TR and ER.

### Phosphatase Experiments

The methods used were as described in Lopez-Molina et al., 2001, above.

RNA extraction, Northern Blots, Antibody Production and Western Analysis

Total RNA extraction was performed as described by Vicient and Delseny, 1999, Anal. Biochem., 268, 412-413. Northern blot hybridizations were by standard procedures; RNA immobilized on membranes was stained with methylene blue and used as a loading control (Sambrook et al., 1989, above). For ABI5, full length ORF DNA probe (SEQ ID NO: 1) were amplified from cDNA with 5′ATGGTAACTAGAGAAACGAAGTTG (SEQ ID NO: 8) 5′ TTAGAGTGGACAACTCGGGTTCCTC (SEQ ID NO: 9).

### Example 1

### ABI5 Activation

The mRNA and protein expression of ABI5 was characterized in WT (Columbia ecotype) seeds at different times after their imbibition.

### ABI5 Expression Under Normal Conditions

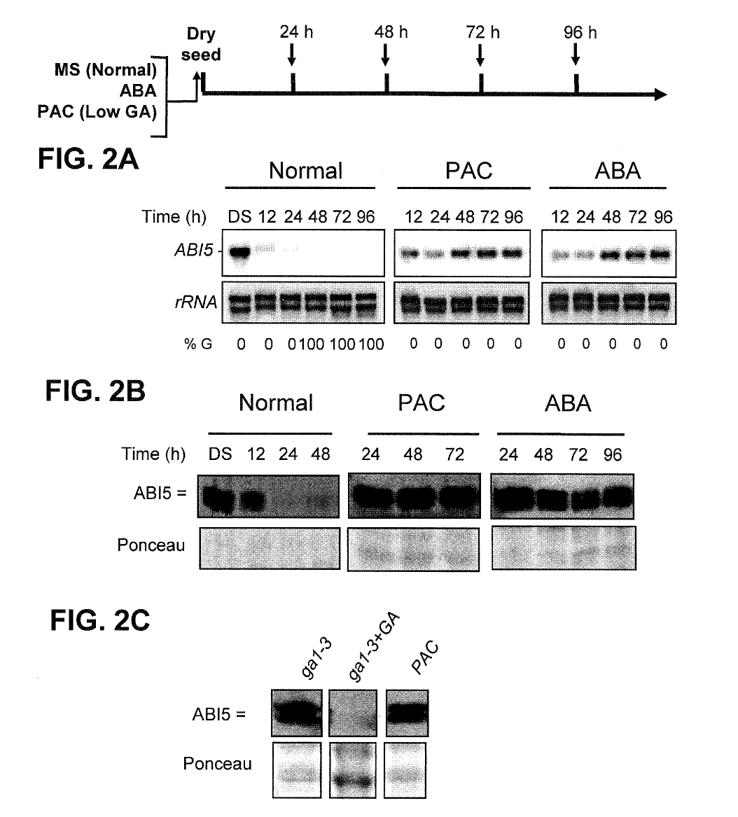

Under normal conditions, ABI5 mRNA and protein levels decreased from peak levels in WT dry seeds (DS) to undetectable levels 24 h to 48 h after imbibition as shown respectively by Northern blot (2 μg of total RNA per lane) and Western blot analyses (10 μg of total protein per lane), where protein extracts were stained with red Ponceau prior to incubation with antibodies against ABI5 (Ponceau) (FIGS. 2A and 2B) (Lopez-Molina et al., 2001, above).

ABI5 mRNA and Protein Expression is Stimulated by Low GA Conditions

ABI5 mRNA and protein expression was stimulated by ABA (5 μM) as previously reported (FIGS. 2A and 2B) (Lopez-Molina et al., 2001, above). Strikingly, low GA conditions (in presence of 5 μM PAC) increased and maintained ABI5 expression for up to 96 hours after imbibition (FIGS. 2A and 2B). The resulting ABI5 mRNA and protein levels were comparable to those observed on ABA conditions. Similarly high ABI5 protein levels were found in gal-3 seeds which are unable to synthesize GA (Koornneet et al., 1983a, Genet. Res., Camb. 41, 57-68), up to 96 hours after imbibition under normal conditions as shown by Western blot analysis (FIG. 2C). ABI5 protein could not be detected in gal-3 seeds treated with GA as early as 48 hours after imbibition (FIG. 2C).

### ABI5 Protein Activity and Phosphorylation in Low GA Conditions

Transgenic lines WT/35S::HA-ABI5 germinate normally (Lopez-Molina et al., 2001, above). However, these lines display germination hypersensitive responses to low ABA concentration (e.g. 0.5 μM), which also triggered HA-ABI5 phosphorylation. This indicated that large amounts of ABI5 protein are not sufficient to repress seed germination (Lopez-Molina et al., 2001, above).

Under low GA conditions (e.g. PAC concentrations at 0.125 μM), the seed germination of WT/35S::HA-ABI5 plants is shown to be hypersensitive and germination arrest (germination of PAC-treated 35S::HA-AB5 seed is strongly delayed: after one week on 0.125 μM PAC, less than 20% of seeds are germinated) is also associated with slower HA-ABI5 migration in SDS-PAGE gels as shown by Western blot analysis from protein extracts isolated from WT/35S::HA-ABI5 seeds (FIGS. 3A and 3B). Phosphatase treatment eliminated the slower migration, as previously reported for ABA, suggesting that it is caused by protein phosphorylation. Thus, low GA levels also stimulate ABI5 activity and phosphorylation. Addition of GA (e.g. 50 μM) in the germination medium did not overcome the repression of seed germination imposed by ABA and did not alter ABA-dependent ABI5 phosphorylation (FIG. 3B). An antibody to HA was used to reveal HA-ABI5 protein. λ phosphatase was inactivated by heat (65° C. for 20 min).

Together, these data suggest that, under normal conditions of germination, ABI5, present at seed imbibition, remains in an inactive form and ABI5 expression is always persistently high in seeds that are unable to germinate, i.e. in conditions of high ABA or low GA. On ABA or low GA conditions, the accumulated ABI5 becomes phosphorylated and activated to repress germination. Conversely, seed germination is always associated with disappearance of ABI5 expression.

### Example 2

### Co-Expression of ABI5 and a Sucrose Non-Fermenting Kinase 1 Related Protein (SnRK2-Type Kinase)

The 35S::HA-ABI5 binary vector (pBA002) was described in Kost et al., 1998, Plant J., 16:393-401 and in Lopez-Molina et al., 2001, above.

PKABA1, encoding a SnRK2-type Ser/Thr kinase from barley was placed under the control of an oestradiol-inducible promoter (ind::HA-PKABA1) such as described on FIG. 4A. Barley PKABA1 cDNA was described by Gomez-Cadenas et al. 1999, Proc. Natl. Acad. Sci. USA, 96, 1767-1772. PKABA10RF DNA sequence of SEQ ID NO: 2 was amplified with the primers:

(SEQ ID NO: 3)

5′CGACTCGAGATGTATCCATATGACGTGCCGGACTACGCCTCCCTCATG
GATCGGTACGAGGTGGTG
and

(SEQ ID NO: 4)

5′CGAACTAGTTCACAACGGGCACACGAAGTC.

The first primer contains an XhoI site and the HA sequence (MYPYDVPDYASL (SEQ ID NO: 5) of SEQ ID NO:10), while the second contains a SpeI site. Both restriction sites were used for cloning into a plasmid pER8 (AN AF309825) such as described in Zuo et al., 2000, Plant J., 24, 265-273. The resulting ind::HA-PKABA1 binary vector was then transformed into previously described WT/35S::HA-ABI5 line (Zuo et al., 2000, above; Lopez-Molina et al., 2001, above). As a negative control, WT plants were transformed with the same ind::HA-PKABA1 construct.

### Western Blot Analysis

Western blot analysis shows similar oestradiol-dependent HA-PKABA1 accumulation 48 h after seed imbibition in WT and WT/35S::HA-ABI5 seeds transformed with the ind::HA-PKABA1 DNA construct under normal conditions (FIG. 4A). HA-PKABA1 protein levels could be detected only in presence of the inducer (50 μM 17β-estradiol) (FIG. 4A). Control lines transformed with the empty inducible vector displayed no additional bands in the presence of the inducer.

The mobility differences of HA-ABI5 protein isolated from WT/35S::HA-ABI5 lines transformed with a ind::HA-PKABA1 DNA construct are analyzed by Western blot under the indicated germination conditions. As observed upon ABA treatment, induction of HA-PKABA1 protein triggered ABI5 phosphorylation, detected as a slower ABI5 protein mobility, which could be eliminated upon phosphatase treatment (FIG. 4B). Excess GA (+) in the medium did not prevent HA-PKABA1-dependent phosphorylation of ABI5 (FIG. 4B).

### Seed Germination

When HA-PKABA1 protein was induced in a WT background, no effect on normal seed germination process could be observed (FIG. 4C). In contrast, inducing HA-PKABA1 protein in WT/35S::HA-ABI5 plants elicited severe delays in seed germination. Similarly to an ABA-imposed germination arrest, additional GA (+) in the medium (e.g. 50 μM) in did not counteract the inducer-imposed arrest (FIG. 4C).

Therefore, co-expression of ABI5 and PKABA1 is sufficient to block seed germination.

Therefore, this system allows in vivo monitoring of the influence of HA-PKABA1 on HA-ABI5 phosphorylation and activity in absence of external manipulations that change endogenous ABA or GA levels.

### Reversibility of Seed Germination Blockage

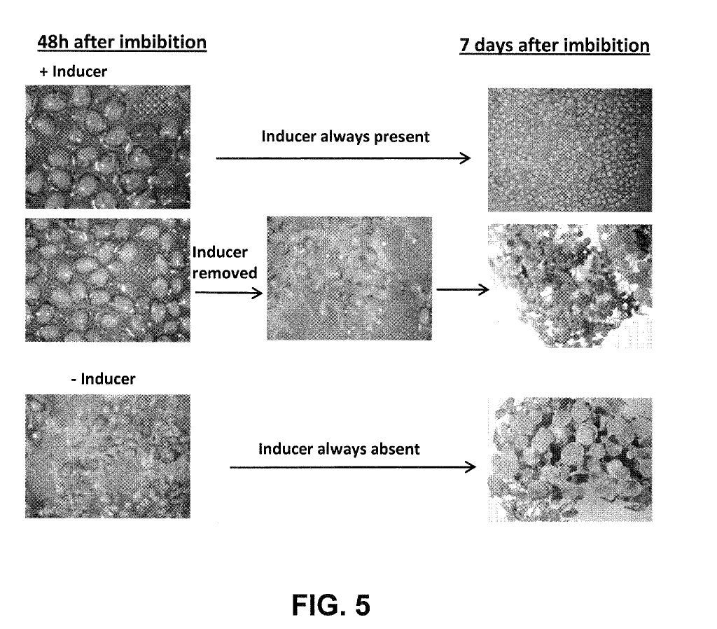

The seed germination blockage in WT plants constitutively expressing HA-ABI5 protein (WT/35S::HA-ABI5) transformed with the ind::HA-PKABA1 DNA construct is reversible as shown by the rapid resumption of seed germination after the removal of the inducer (50 μM 17β estradiol) by a medium shift as seeds are plated on a permeable support such as nylon or whatmann paper so that they can be transferred at will. (FIG. 5): 24 h to 56 h upon transfer, 100% germination could be observed soon followed by greening and normal seedling growth, which was unlike plants kept in presence of the inducer. Therefore, the germination reaction is reversible and depends on the concentration of phosphorylated ABI5 in plants.

### Properties of the Transgenic Seeds

The reversibility of seed germination blockage as observed above, suggests that the nutritional properties of the seeds are preserved as food stores useful to fuel seed germination are functional and can be used when germination process is reactivated.

Further the transgenic seeds show preserved osmotolerance properties such as observed by as described in Lopez-Molina et al., 2001, above.

### Expression of AtEm6 is Induced by Phosphorylated ABI5

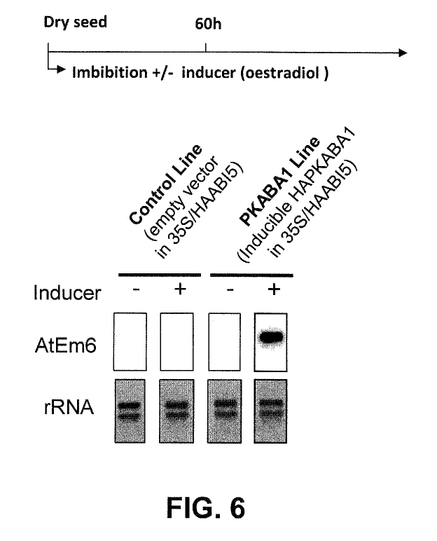

The induction of PKABA1 in WT/35S:: HA-ABI5 plants increases the expression of osmototerance gens such as Late Embryonic and Abundant genes (LEA) as assessed by the strong accumulation of AtEm6 (FIG. 6) and AtEm1 transcripts which are dependent on the active ABI5.

Taken together, the data show that all the germination responses observed under ABA conditions can be mimicked in vivo by co-expressing HA-PKABA1, via an inducible transgene, and HA-ABI5, via a constitutive transgene. They indicate that ABA-dependent ABI5 activation to repress germination may involve a SnRK2-type kinase activity phosphorylating ABI5.

We claim:

1. A method of producing a transgenic plant, comprising the steps of:
(a) transforming a plant cell with: (i) a first expression cassette comprising a promoter operably linked to a nucleotide sequence encoding for absisic acid insensitive 5 (ABI5); and (ii) a second expression cassette comprising an inducible promoter operably linked to a nucleotide sequence encoding for a Sucrose non-fermenting 1-related protein kinase 2 type kinase (SnRK2-type kinase);
(b) regenerating from said transformed plant cell a genetically transformed plant.

2. The method according to claim 1, wherein the promoter of said first expression cassette is a constitutive promoter and the promoter of said second expression cassette is an estradiol inducible promoter.

3. The method according to claim 1, wherein the constitutive promoter is a 35S promoter and the inducible promoter is a XVE promoter.

4. A method of producing a transgenic seed with delayed germination, as compared to wild-type plant comprising the steps of:
(a) transforming a plant cell with: (i) a first expression cassette comprising a promoter operably linked to a nucleotide sequence encoding for ABI5; and a (ii) second expression cassette comprising an inducible promoter operably linked to a nucleotide sequence encoding for a SnRK2-type kinase;
(b) regenerating from said transformed plant cell a genetically transformed plant; and
(c) collecting a population of transgenic seeds from said transgenic plant.

5. The method according to claim 4, wherein the promoter of said first expression cassette is a constitutive promoter and the promoter of said second expression cassette is an estradiol inducible promoter.

6. The method according to claim 4, wherein the constitutive promoter is a 35S promoter and the inducible promoter is a XVE promoter.

7. A method of controlling seed germination, comprising:
(a) providing a seed having a genome comprising a first stably integrated expression cassette wherein said expression cassette comprises a nucleotide sequence encoding for a promoter which is operably linked to a nucleotide sequence encoding for ABI5 and (ii) a second stably integrated expression cassette wherein said expression cassette comprises a nucleotide sequence of an inducible promoter operably linked to a nucleotide sequence encoding for a SnRK2-type kinase; and
(b) inducing the inducible promoter to block seed germination.

8. The method according to claim 7, wherein the promoter of said first expression cassette is a constitutive promoter and the promoter of said second expression cassette is an estradiol inducible promoter.

9. The method according to claim 7, wherein the constitutive promoter is a 35S promoter and the inducible promoter is a XVE promoter.

10. A composition of matter comprising:
a) a plant and the progeny thereof, which comprises a first stably integrated expression cassette wherein said expression cassette comprises a nucleotide sequence encoding for a promoter which is operably linked to a nucleotide sequence encoding for ABI5 and (ii) a second stably integrated expression cassette wherein said expression cassette comprises a nucleotide sequence of an inducible promoter operably linked to a nucleotide sequence encoding for a SnRK2-type kinase;
b) a transgenic seed comprising a DNA construct capable of (i) constitutively expressing functional ABI5 and of (ii) expressing for a SnRK2-type kinase under induction, at least during the period of seed maturation;
c) a transgenic plant or seed obtainable by a method according to claim 1;
d) a transgenic plant or seed obtainable by a method according to claim 4;
e) a transgenic plant or seed obtainable by a method according to claim 7;
f) an expression cassette comprising a promoter operably linked to (i) a DNA sequence encoding a DNA-binding domain of LexA (residues 1-87); (ii) a DNA sequence encoding a transcription activation domain of VP16 (residues 403-479); (iii) a DNA sequence encoding a regulatory region of an estrogen receptor and (iv) an amplified PKABA1 DNA into the Xho1 and Spe1 restriction sites, wherein the PKABA1 DNA sequence comprises a silent selectable marker;
g) an expression cassette comprising a promoter operably linked to (i) a DNA sequence encoding a DNA-binding domain of LexA (residues 1-87); (ii) a DNA sequence encoding a transcription activation domain of VP16 (residues 403-479); (iii) a DNA sequence encoding a regulatory region of an estrogen receptor and (iv) an amplified PKABA1 DNA into the Xho1 and Spe1 restriction sites, wherein the PKABA1 DNA sequence comprises a silent selectable marker, wherein the amplified PKABA1 DNA is HA-PKABA1 DNA;
h) a recombinant vector comprising an expression cassette comprising an expression cassette comprising a promoter operably linked to (i) a DNA sequence encoding a DNA-binding domain of LexA (residues 1-87); (ii) a DNA sequence encoding a transcription activation domain of VP16 (residues 403-479); (iii) a DNA sequence encoding a regulatory region of an estrogen receptor and (iv) an amplified PKABA1 DNA into the Xho1 and Spe1 restriction sites, wherein the PKABA1 DNA sequence comprises a silent selectable marker;
i) a transgenic plant cell comprising an expression cassette comprising a promoter operably linked to (i) a DNA sequence encoding a DNA-binding domain of LexA (residues 1-87); (ii) a DNA sequence encoding a transcription activation domain of VP16 (residues 403-479); (iii) a DNA sequence encoding a regulatory region of an estrogen receptor and (iv) an amplified PKABA1 DNA into the Xho1 and Spe1 restriction sites, wherein the PKABA1 DNA sequence comprises a silent selectable marker; or
j) a transgenic plant comprising a transgenic plant cell comprising an expression cassette comprising a promoter operably linked to (i) a DNA sequence encoding a DNA-binding domain of LexA (residues 1-87); (ii) a DNA sequence encoding a transcription activation domain of VP16 (residues 403-479); (iii) a DNA sequence encoding a regulatory region of an estrogen receptor and (iv) an amplified PKABA1 DNA into the Xho1 and Spe1 restriction sites, wherein the PKABA1 DNA sequence comprises a silent selectable marker.

11. A method of producing a crop, said method comprising: planting a transgenic plant or a seed according to claim 10 and harvesting a resulting crop.

12. An agricultural product produced by a transgenic plant or by a transgenic seed according to claim 10.
